(19) United States
(12) Patent Application Publication
(10) Pub. No.: US 2005/0209319 A1
(43) Pub. Date: Sep. 22, 2005

(54) Treatment of local pain

(75) Inventor: Kenneth C. Cundy, Redwood City, CA (US)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: XenoPort, Inc., Santa Clara, CA (US)

(21) Appl. No.: 11/079,678

(22) Filed: Mar. 15, 2005

Related U.S. Application Data
(60) Provisional application No. 60/554,582, filed on Mar. 18, 2004.

(51) Int. Cl. A61K 31/325; A61K 31/215; A61K 31/195

(52) U.S. Cl. 514/484; 514/529; 514/561

(57) ABSTRACT

Methods and compositions for treating or preventing local pain or discomfort, particularly local neuropathic pain via topical application directly to skin or mucosal tissue at the site of pain or discomfort are disclosed. Compositions comprising prodrugs of gamma amino butyric acid analogs, such as prodrugs of gabapentin or pregabalin, and optionally a topical anesthetic agent are also disclosed.

This application claims benefit of priority to U.S. Provisional Application No. 60/554,582 filed Mar. 18, 2004, which is incorporated by reference herein in its entirety.

Disclosed herein are methods and compositions for treating or preventing pain or discomfort via topical application when applied to a site of local pain or discomfort such as on the skin. The disclosed methods and compositions can produce a local analgesic effect and have particular utility in treating localized neuropathic pain.

Pain results from the noxious stimulation of nerve endings. Nociceptive pain is caused by noxious stimulation of nociceptors (e.g., a needle stick or skin pinch), which then transmit impulses over intact neural pathways to the spinal nerves and then to the brain. Neuropathic pain is a form of chronic pain that can persist for months, years, or decades following an injury or a viral infection such as herpes zoster (shingles) and typically results from damage to peripheral nerves, nerve roots, the spinal cord or certain brain regions. See the discussion of neuropathic pain in Sawynok et al., U.S. Pat. No. 6,211,171, columns 1 through 3, the disclosures of which are incorporated herein by reference. Neuropathic pain is caused by damage to neural structures, such as damage to peripheral nerve endings or nociceptors, which become extremely sensitive to stimulation and can generate impulses in the absence of stimulation (e.g., herpes zoster pain after the rash has healed or phantom limb pain). Peripheral nerve damage can lead to pathological states where the pain threshold is reduced (i.e., allodynia), an increased response to noxious stimuli (hyperalgesia), or a prolonged response duration (chronic pain). There are two broad types of neuropathic pain: deafferentation pain (caused by partial or complete interruption of central or peripheral neural activity) and sympathetically maintained pain (due to efferent sympathetic activity) (See “Neuropathic Pain,” in The Merck Manual, 17^{th} Ed., M. H. Beers and R. Berkow, eds., Merck Research Laboratories, Whitehouse Station, N.J., 1999, pp. 1371-1372). Examples of neuropathic pain include diabetic neuropathy, postherpatic neuralgia and chronic musculoskeletal disorders.

In contrast to pain treatment with systemic agents, neuropathic pain can be treated locally by topically administering a local anesthetic directly to the painful area to block the transmission of the painful sensation. Local anesthetics prevent the generation and conduction of nociceptive nerve impulses and therefore can be effective against both nociceptive and neuropathic pain. Thus, for example, a local anesthetic can be injected intradermally (non-systemic injection within the skin) or topically applied at the painful area. Advantages of topical local anesthetic administration over systemic administration of pain relievers include decrease or preclusion of side effects, improved patient compliance, and reversible action (i.e., the action can be reversed by removing the anesthetic from the application site). The principal disadvantage of local anesthetic administration in treating severe neuropathic pain is the difficulty in getting sufficient concentrations of the active agent through the dermal barrier.

Gabapentin is a structural analog of gamma-aminobutyric acid (GABA) with demonstrated therapeutic utility in epilepsy, post-herpetic neuralgia, diabetic neuropathy, pain in Guillain-Barre syndrome, post-amputation phantom limb pain, neuropathic pain after spinal cord injury, essential tremor, Parkinson's disease and syndrome, restless legs syndrome, amyotrophic lateral sclerosis and post-menopausal hot flashes (See Magnus, Epilepsia, 1999, 40, S66-72). The mechanism of action of gabapentin in most indications remains undefined, but the drug does not directly interact with GABA receptors. Gabapentin binds to the alpha-2-delta subunit of the voltage-dependent calcium channel (See Dissanayake et al., Br. J. Pharmacol., 1997, 120:5, 833-40). Gabapentin, when administered orally and systemically, has shown utility in treating or preventing neuropathic pain (See for example, Backonja et al., Clin. Ther., 2003, 25(1), 81-104; Mellegers et al., Clin. J. Pain, 2001, 17, 284-295).

Gabapentin and a related GABA analog, pregabalin, are both conventionally dosed as oral formulations (tablets, capsules and oral solution) for systemic administration.

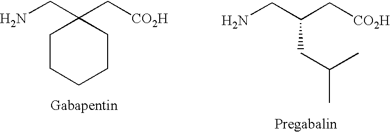

In addition to gabapentin and pregabalin, a number of other gamma amino butyric acid analogs have been disclosed for the systemic treatment of neuropathic pain. See for example, Bryans et al., U.S. Pat. No. 6,245,801; Bryans et al., U.S. Pat. No. 6,316,638; Belliotti, et al., U.S. Pat. No. 6,436,974; Bryans et al., U.S. Pat. No. 6,489,352; Bryans et al., U.S. Pat. No. 6,518,289; Belliotti, et al., U.S. Pat. No. 6,521,650; Bryans et al., U.S. Pat. No. 6,545,022; Blakemore et al., U.S. Pat. No. 6,596,900; Bryans et al., U.S. Patent Application Publication No. 2002/0019540; Blakemore et al., U.S. Patent Application Publication No. 2003/0078300; and Bryans et al., U.S. Patent Application Publication No. 2003/0119858. The daily systemic dose of gabapentin for treating neuropathic pain typically ranges from about 300 mg to about 2400 mg with many patients requiring doses above 1500 mg per day. Early published reports on the clinical testing of pregabalin suggest that the required oral daily systemic dose of pregabalin ranges from about 500 mg to about 1800 mg. Such high daily doses of gabapentin and pregabalin make the compounds unattractive candidates for transdermal systemic administration. This is because transdermal patches and plasters (transdermal delivery systems) can typically administer no more than about 20 mg of drug per day (for example, NicodermCQ® transdermal system delivers up to about 21 mg of nicotine per day; Duragesic® transdermal system delivers up to about 2.5 mg of fentanyl per day; NitroDur® transdermal system delivers up to about 19 mg of nitroglycerin per day; and Testoderm® transdermal system delivers up to about 6 mg of testosterone per day). Furthermore, the physicochemical properties of gabapentin and pregabalin suggest that these compounds are poor candidates for transdermal systemic administration.

In addition to systemic administration, there have been disclosures of local administration of gabapentin in treating pain. For example, Carlton and Zhou have shown that subcutaneous injection of gabapentin has a peripheral effect in an animal model of pain (Carlton and Zhou, Pain, 1998, 76 (1-2), pp. 201-7). Gabapentin was injected into the hindpaw of rats that had been pretreated with a 2% formalin injection in order to induce localized pain. The gabapentin injection significantly reduced nociceptive behavior (i.e., the animal's typical behavior upon perceiving pain). This antihyperalgesic effect was not due to a central action, since direct injection of gabapentin had no effect on the animals' nociceptive behavior following formalin injection into the contralateral (i.e., the animals' other) hindpaw. The antihyperalgesic effect of gabapentin was also not due to a local anesthetic effect, since needle sticks within the drug-injected region evoked paw withdrawal behavior that was not different from pre-drug levels. Pregabalin was shown to have a similar local effect on nociception in this model.

The topical application of gabapentin in combination with other drug(s) such as carbemazipine, ketoprofen and others in treating pain has been disclosed. Murdock et al. in U.S. Pat. No. 6,572,980 disclose topically applied pastes containing gabapentin and at least one other drug such as ketoprofen, prioxicam, carbamazepine, doxepin and guaifenesin. These pastes exhibit inconsistent therapeutic benefits in treating patients suffering from localized pain of various origins.

Local anesthetics have also been used as topical treatments for local neuropathic pain. Hind in U.S. Pat. No. 5,411,738 discloses the use of topically applied lidocaine in the treatment of post-herpetic neuropathic pain. Gammaitoni et al. report that the Lidoderm® transdermal system, a patch having a non-woven polyester felt backing and a skin-contacting adhesive containing 5% lidocaine, shows efficacy in the treatment of various peripheral neuropathic pain states (Gammaitoni et al., J. Clin. Pharmacol., 2003, 43(2), pp. 111-117). The most common adverse events for the Lidoderm® patch generally involve mild skin reactions.

Disclosed herein are methods and topical compositions for treating, reducing or preventing local pain or discomfort, including local pain or discomfort, and in particular, neuropathic pain. Compositions of the present disclosure can be topically administered to the skin or mucosa of a subject to provide local pain-reducing or local pain-eliminating effect, e.g., a local analgesic effect.

Certain aspects of the present disclosure provide topical compositions for treating or preventing pain or discomfort comprising a compound chosen from Formula (1) and Formula (2): 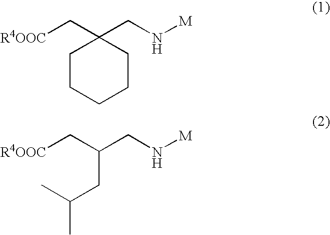
wherein:
   R⁴ is chosen from hydrogen and a labile ester-forming group chosen from C₁₋₆ alkyl, benzyl, and phenyl groups that become removed in the body of a subject;
   M is a moiety that becomes removed in the body of a subject and which increases skin permeability of the compound to a level greater than the skin permeability of a modified compound formed by replacing either M or both M and R⁴ with hydrogen;
or a pharmaceutically acceptable salt, hydrate or solvate thereof; and
   a pharmaceutically acceptable vehicle.

Certain aspects of the present disclosure provide topical compositions comprising a compound chosen from Formula (1) and Formula (2) or a pharmaceutically acceptable salt, hydrate or solvate thereof, a pharmaceutically acceptable vehicle, and a local anesthetic.

Certain aspects of the present disclosure provide methods of treating or preventing pain or discomfort in a subject having a site of local pain or discomfort, comprising locally administering to the site a therapeutically effective amount of a compound chosen from Formula (1) and Formula (2): 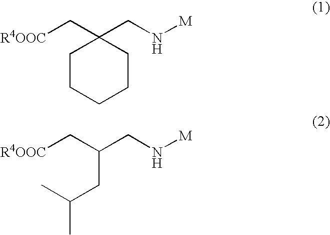
wherein:
   R⁴ is chosen from hydrogen and a labile ester-forming group chosen from C₁₋₆ alkyl, benzyl, and phenyl groups that become removed in the body of a subject;
   M is a moiety that becomes removed in the body of a subject and which increases skin permeability of the compound to a level greater than the skin permeability of a modified compound formed by replacing either M or both M and R⁴ with hydrogen;
      or a pharmaceutically acceptable salt, hydrate or solvate thereof.

Additional embodiments of the invention are set forth in the description which follows, or may be learned by practice of the invention.

Definitions Used in the Present Disclosure

As used in the present specification, the following words and phrases are generally intended to have the meanings as set forth below, except to the extent that the context in which they are used indicates otherwise.

As used herein, when any variable occurs more than one time in a chemical formula, its definition on each occurrence is independent of its definition at every other occurrence. In accordance with the usual meaning of “a” and “the” in patents, reference to “a” compound or “the” compound is inclusive of one or more compounds. Unless otherwise specified the terms “compound” and “compounds” include all pharmaceutically acceptable forms of the disclosed structures salts, hydrates, solvates and the like.

“Alkyl” by itself or as part of another substituent refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. In certain embodiments, an alkyl group comprises from 1 to 6 carbon atoms.

“Compounds” as used herein refers to GABA analogs and prodrugs of GABA analogs, and includes any specific compounds encompassed by generic formulae disclosed herein. The compounds may be identified either by their chemical structure and/or chemical name. When the chemical structure and chemical name conflict, the chemical structure is determinative of the identity of the compound. The compounds may contain one or more chiral centers and/or double bonds and therefore, may exist as stereoisomers, such as double-bond isomers (i.e., geometric isomers), enantiomers or diastereomers. Accordingly, when stereochemistry at chiral centers is not specified, the chemical structures depicted herein encompass all possible configurations at those chiral centers including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated compounds. The compounds also include isotopically labeled compounds where one or more atoms have an atomic mass different from the atomic mass conventionally found in nature. Examples of isotopes that may be incorporated into the compounds include, but are not limited to, ²H, ³H, ¹³C, ¹⁴C, ¹⁵N, ¹⁷O, and ¹⁸O. Compounds may exist in unsolvated forms as well as solvated forms, including hydrated forms. In general, the hydrated, solvated and unsolvated forms are within the scope of the present disclosure. Certain compounds may exist in multiple crystalline or amorphous forms. In general, all physical forms are equivalent for the uses contemplated herein and are intended to be within the scope of the present disclosure. Further, it should be understood, when partial structures of the compounds are illustrated, that brackets indicate the point of attachment of the partial structure to the rest of the molecule.

“Labile” and “become removed” are used synonymously herein to describe the in vivo cleavability of certain moieties of a GABA analog prodrugs. The disclosed prodrugs can be metabolized to form the parent GABA analog (e.g., gabapentin or pregabalin) following administration to a patient. The promoiety, M, and the group R⁴ of the GABA analog prodrugs can be cleaved either chemically and/or enzymatically. For example, one or more enzymes present in a local tissue of a patient at the site of topical application can enzymatically cleave the moiety M, and when R⁴ is other than hydrogen, can also cleave R⁴, to release the parent GABA analog drug. The cleavability of M and R⁴ can produce therapeutically effective amounts of a parent GABA analog drug in a local tissue of a patient. Those skilled in the art will appreciate that the required level of cleavability of M and R⁴ can depend on a number of factors, including the potency and half-life of a particular parent GABA analog.

“Local anesthetic” means any drug that provides local numbness or loss of sensation.

“Pharmaceutically acceptable salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and (2) salts formed when an acidic proton present in the parent compound is replaced by a metal ion, for example, an alkali metal ion, an alkaline earth ion, or an aluminum ion; or coordinates with an organic base such as ethanolamine, diethanolamine, triethanolamine, N-methylglucamine, and the like.

“Pharmaceutically acceptable vehicle” refers to a diluent, adjuvant, excipient or carrier with which a compound is administered.

“Pharmaceutical composition” as used herein refers to a GABA analog prodrug, an optional local anesthetic, an optional vasoconstrictor and a pharmaceutically acceptable vehicle with which the prodrug is administered to a patient.

“Prevent”, “preventing” and “prevention” of pain means (1) reducing the risk of a patient who is not experiencing pain from developing pain, or (2) reducing the frequency of, the severity of, or a complete elimination of, pain already being experienced by a patient.

“Prodrug” refers to a derivative of a drug molecule that requires a transformation, e.g., metabolism within the body to release the active drug/metabolite.

“Promoiety” means a chemical moiety M which when covalently bound to a parent drug molecule converts the parent drug into a prodrug. The promoiety M is attached to the parent drug via bond(s) that can be cleaved by enzymatic or non-enzymatic means in vivo. Examples of promoieties include those that effect increased transdermal absorption of a prodrug following topical administration relative to the topical administration of the parent drug at an equimolar dose.

“Subject” includes humans and animals. The terms “subject” and “patient” are used interchangeably herein.

“Therapeutically effective amount” means the amount of a compound that, when administered to a patient for treating or preventing pain or discomfort, is sufficient to effect such treatment or prevention of the pain or discomfort. A “therapeutically effective amount” can vary depending, for example, on the compound, the severity of the pain or discomfort, the etiology of the pain or discomfort, the age of the patient to be treated and/or the weight of the patient to be treated.

“Topical” means the delivery of a pharmacologically active agent to the skin or mucosa of a patient. Topical administration can provide a local rather than a systemic effect. The terms “topical administration” and “transdermal administration” are used interchangeably to mean administration of a pharmacologically active agent to the skin or mucosa of a patient to achieve a therapeutic effect in treating or preventing pain or discomfort at the site of topical or transdermal administration.

“Treat”, “treating” and “treatment” of pain or discomfort means reducing the frequency of symptoms of pain or discomfort, eliminating the symptoms of pain or discomfort, avoiding or arresting the development of pain or discomfort, and/or reducing the severity of symptoms of pain or discomfort.

Reference will now be made in detail to embodiments of the present disclosure. While certain embodiments of the present disclosure will be described, it will be understood that it is not intended to limit the embodiments of the present disclosure to those described herein. To the contrary, reference to embodiments of the present disclosure is intended to cover alternatives, modifications, and equivalents as may be included within the spirit and scope of the embodiments of the present disclosure by the appended claims.

Description of Certain Embodiments

The drugs gabapentin and pregabalin are thought to have a peripheral site of action in addition to their central nervous system activity. However, the physicochemical properties of gabapentin and pregabalin suggest that these compounds would have limited transdermal absorption following topical administration. Gabapentin has limited passive permeability in vitro across artificial membranes as demonstrated by PAMPA assays and through Caco-2 cell monolayers. In contrast, prodrugs of gabapentin with greater passive permeability can have significant transdermal flux when applied topically to the skin. For example, prodrug 1-{[(α-isobutanoyl-oxyethoxy)carbonyl]-aminomethyl}-1-cyclohexane acetic acid exhibits relatively high passive permeability in vitro across artificial membranes (PAMPA assays) and through Caco-2 cell monolayers. The methods and compositions disclosed herein contemplate the use of prodrugs of gabapentin, prodrugs of pregabalin or related GABA analogs with significant passive permeability sufficient to provide therapeutically useful local exposure to gabapentin.

Certain embodiments of the present disclosure provide topical compositions for treating or preventing pain or discomfort comprising a compound chosen from Formula (1) and Formula (2): 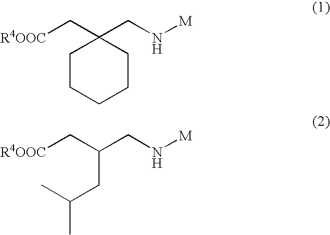
wherein:
   R⁴ is chosen from hydrogen and a labile ester-forming group chosen from C₁₋₆ alkyl, benzyl, and phenyl groups that become removed in the body of a subject;
   M is a moiety that becomes removed in the body of a subject and which increases skin permeability of the compound to a level greater than the skin permeability of a modified compound formed by replacing either M or both M and R⁴ with hydrogen;
or a pharmaceutically acceptable salt, hydrate or solvate thereof; and
   a pharmaceutically acceptable vehicle.

In certain embodiments of compounds of Formula (1) and Formula (2), M is a moiety of Formula (3): 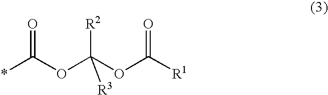
wherein:
   R¹ is chosen from C₁₋₆ alkyl, and 1,1-diethoxyethyl; and
   R² and R³ are independently chosen from hydrogen, and C₁₋₆ alkyl.

In certain embodiment of compounds of Formula (1) and Formula (2), M is a moiety of Formula (3): 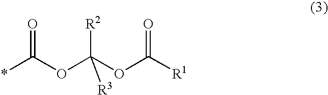
wherein:
   R¹ is selected from the group consisting of methyl, ethyl, propyl, isopropyl, butyl, isobutyl, sec-butyl, tert-butyl, pentyl, isopentyl, sec-pentyl, neopentyl, and 1,1-diethoxyethyl; and
   R² and R³ are independently chosen from hydrogen, methyl, ethyl, propyl, isopropyl, butyl, isobutyl, and sec-butyl.

In certain embodiments of a compound of Formula (2), the compound is a substantially pure optical isomer of Formula (4):

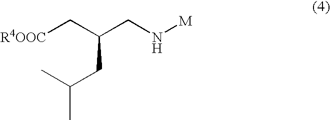

In certain embodiments of compounds chosen from Formula (1) and Formula (2), moiety M can increase the artificial membrane permeability coefficient (“Pₐₘ”) of the compound to a level that is at least 3 times, 5 times, or 7 times greater than the Pₐₘ of the parent GABA analog, i.e, the modified compound, using artificial membrane permeability assays as disclosed herein.

In certain embodiments of compounds chosen from Formula (1) and Formula (2), moiety M can increase the apparent permeability coefficient (“Pₐₚₚ”) of the compound to a level that is at least 25%, 50%, or 75% greater than the Pₐₚₚ of the parent GABA analog, i.e, the modified compound, using Caco-2 permeability assays as disclosed herein.

In certain embodiments, a compound of Formula (1) is chosen from:
1-{[(α-acetoxyethoxy)carbonyl]aminomethyl}-1-cyclohexane acetic acid;
1-{[(α-propanoyloxyethoxy)carbonyl]aminomethyl}-1-cyclohexane acetic acid;
1-{[(α-butanoyloxyethoxy)carbonyl]aminomethyl}-1-cyclohexane acetic acid;
1-{[(α-isobutanoyloxyethoxy)carbonyl]aminomethyl}-1-cyclohexane acetic acid;
1-{[(α-pivaloxyethoxy)carbonyl]aminomethyl}-1-cyclohexane acetic acid;
1-{[(α-acetoxymethoxy)carbonyl]aminomethyl}-1-cyclohexane acetic acid;
1-{[(α-propanoyloxymethoxy)carbonyl]aminomethyl}-1-cyclohexane acetic acid;
1-{[(α-butanoyloxymethoxy)carbonyl]aminomethyl}-1-cyclohexane acetic acid;
1-{[(α-isobutanoyloxymethoxy)carbonyl]aminomethyl}-1-cyclohexane acetic acid;
1-{[(α-pivaloxymethoxy)carbonyl]aminomethyl}-1-cyclohexane acetic acid;
1-{[(α-acetoxypropoxy)carbonyl]aminomethyl}-1-cyclohexane acetic acid;
1-{[(α-propanoyloxypropoxy)carbonyl]aminomethyl}-1-cyclohexane acetic acid;
1-{[(α-butanoyloxypropoxy)carbonyl]aminomethyl}-1-cyclohexane acetic acid;
1-{[(α-isobutanoyloxypropoxy)carbonyl]aminomethyl}-1-cyclohexane acetic acid;
1-{[(α-pivaloxypropoxy)carbonyl]aminomethyl}-1-cyclohexane acetic acid;
1-{[(α-acetoxyisopropoxy)carbonyl]aminomethyl}-1-cyclohexane acetic acid;
1-{[(α-propanoyloxyisopropoxy)carbonyl]aminomethyl}-1-cyclohexane acetic acid;
1-{[(α-butanoyloxyisopropoxy)carbonyl]aminomethyl}-1-cyclohexane acetic acid;
1-{[(α-isobutanoyloxyisopropoxy)carbonyl]aminomethyl}-1-cyclohexane acetic acid;
1-{[(α-pivaloxyisopropoxy)carbonyl]aminomethyl}-1-cyclohexane acetic acid;
1-{[(α-acetoxybutoxy)carbonyl]aminomethyl}-1-cyclohexane acetic acid;
1-{[(α-propanoyloxybutoxy)carbonyl]aminomethyl}-1-cyclohexane acetic acid;
1-{[(α-butanoyloxybutoxy)carbonyl]aminomethyl}-1-cyclohexane acetic acid;
1-{[(α-isobutanoyloxybutoxy)carbonyl]aminomethyl}-1-cyclohexane acetic acid; and
1-{[(α-pivaloxybutoxy)carbonyl]aminomethyl}-1-cyclohexane acetic acid.

In certain embodiments, a compound of Formula (1) is 1-{[α-isobutanoyloxyethoxy)carbonyl]-aminomethyl}-1-cyclohexane acetic acid.

In certain embodiments, a compound of Formula (2) is chosen from:
3-{[(α-acetoxyethoxy)carbonyl]aminomethyl}-5-methyl hexanoic acid;
3-{[(α-propanoyloxyethoxy)carbonyl]aminomethyl}-5-methyl hexanoic acid;
3-{[(α-butanoyloxyethoxy)carbonyl]aminomethyl}-5-methyl hexanoic acid;
3-{[(α-isobutanoyloxyethoxy)carbonyl]aminomethyl}-5-methyl hexanoic acid;
3-{[(α-pivaloxyethoxy)carbonyl]aminomethyl}-5-methyl hexanoic acid;
3-{[(α-acetoxymethoxy)carbonyl]aminomethyl}-5-methyl hexanoic acid;
3-{[(α-propanoyloxymethoxy)carbonyl]aminomethyl}-5-methyl hexanoic acid;
3-{[(α-butanoyloxymethoxy)carbonyl]aminomethyl}-5-methyl hexanoic acid;
3-{[(α-isobutanoyloxymethoxy)carbonyl]aminomethyl}-5-methyl hexanoic acid;
3-{[(α-pivaloxymethoxy)carbonyl]aminomethyl}-5-methyl hexanoic acid;
3-{[(α-acetoxypropoxy)carbonyl]aminomethyl}-5-methyl hexanoic acid;
3-{[(α-propanoyloxypropoxy)carbonyl]aminomethyl}-5-methyl hexanoic acid;
3-{[(α-butanoyloxypropoxy)carbonyl]aminomethyl}-5-methyl hexanoic acid;
3-{[(α-isobutanoyloxypropoxy)carbonyl]aminomethyl}-5-methyl hexanoic acid;
3-{[(α-pivaloxypropoxy)carbonyl]aminomethyl}-5-methyl hexanoic acid;
3-{[(α-acetoxyisopropoxy)carbonyl]aminomethyl}-5-methyl hexanoic acid;
3-{[(α-propanoyloxyisopropoxy)carbonyl]aminomethyl}-5-methyl hexanoic acid;
3-{[(α-butanoyloxyisopropoxy)carbonyl]aminomethyl}-5-methyl hexanoic acid;
3-{[(α-isobutanoyloxyisopropoxy)carbonyl]aminomethyl}-5-methyl hexanoic acid;
3-{[(α-pivaloxyisopropoxy)carbonyl]aminomethyl}-5-methyl hexanoic acid;
3-{[(α-acetoxybutoxy)carbonyl]aminomethyl}-5-methyl hexanoic acid;
3-{[(α-propanoyloxybutoxy)carbonyl]aminomethyl}-5-methyl hexanoic acid;
3-{[(α-butanoyloxybutoxy)carbonyl]aminomethyl}-5-methyl hexanoic acid;
3-{[(α-isobutanoyloxybutoxy)carbonyl]aminomethyl}-5-methyl hexanoic acid; and
3-{[(α-pivaloxybutoxy)carbonyl]aminomethyl}-5-methyl hexanoic acid.

In certain embodiments, a composition of the present disclosure can comprise more than one compound chosen from Formula (1) and Formula (2).

Methods of synthesizing compounds of Formulae (1) and (2) are known and disclosed, for example, in Gallop et al., International Publication No. WO 02/100347. Methods for synthesizing other GABA analog prodrugs are disclosed, for example, in Bryans et al., International Publication No. WO 01/90052; U.K. Application No. GB 2,362,646; European Application Nos. EP 1,201,240 and EP 1,178,034; Yatvin et al., U.S. Pat. No. 6,024,977; Gallop et al., International Publication No. WO 02/28881; Gallop et al., International Publication No. WO 02/28883; Gallop et al., International Publication No. WO 02/28411; Gallop et al., International Publication No. WO 02/32376; and Gallop et al., International Publication No. WO 02/42414), the disclosures of which are incorporated herein by reference.

When used in the present methods of treatment, a prodrug of a GABA analog can be applied topically, and can be metabolized to release a parent GABA analog (e.g., gabapentin or pregabalin) at or about the peripheral site of application of the prodrug in a therapeutically effective amount. A promoiety or promoieties of a GABA analog prodrug can be cleaved either chemically and/or enzymatically within the skin or mucosal tissue of the patient at the local site of application of the prodrug. The mechanism of cleavage is not critical to the therapeutic methods disclosed herein. In certain embodiments, the cleaved promoiety or promoieties and any metabolites thereof have low toxicity. As disclosed in Augart et al., U.S. Pat. No. 6,054,482, certain GABA analogs such as gabapentin can cyclize (by reaction of the carboxylic group with the amino group) to form a lactam. In the case of gabapentin, the lactam is considered an undesirable impurity. Hence, in certain embodiments, a prodrug of a GABA analog of the present disclosure can metabolize to form the parent GABA analog without forming substantial quantities of the corresponding lactam. In certain embodiments, less than 5% of the administered prodrug is metabolized to form a GABA lactam, in certain embodiments less than 1%, and in certain embodiments less than 0.5%. The extent of lactam formation from the metabolism of a prodrug of a GABA analog can be assessed using standard in vitro analytical methods. For similar reasons, in certain embodiments, GABA analog prodrugs disclosed herein are stable during storage and do not form substantial quantities of lactam impurities, for example, by cyclization of the GABA analog and/or GABA analog prodrug during storage.

In certain embodiments, a composition for topical administration can comprise a prodrug of a GABA analog and a local anesthetic agent. Local anesthetics can provide enhanced relief of local pain. Topical delivery of a prodrug of a GABA analog and a local anesthetic can provide minimal risk of systemic toxicity and/or adverse drug-drug interactions. Although the mechanisms of action of GABA analogs such as gabapentin and pregabalin and local anesthetics such as lidocaine are believed to be significantly different, the disclosed methods and compositions can provide effective therapeutic treatment of local pain or discomfort, particularly local neuropathic pain.

Examples of local anesthetics suitable for use in the compositions and methods of the present disclosure include ambucaine, amolanone, amylcaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dyclonine, ecogonidine, ecogonine, euprocin, fenalcomine, formocaine, hexylcaine, hydroxyteteracaine, isobutyl p-aminobenzoate, leucinocaine, levoxadrol, lidocaine, mepivacaine, meprylcaine, metabutoxycaine, methyl chloride, myrtecaine, naepaine, octacaine, orthocaine, oxethazaine, parenthoxycaine, phenacaine, phenol, piperocaine, piridocaine, polidocanol, pramoxine, prilocalne, procaine, propanocaine, proparacaine, propipocaine, propoxycaine, pseudococaine, pyrrocaine, ropivacaine, salicyl alcohol, tetracaine, tolycaine, trimecaine, zolamine, and pharmaceutically acceptable salts thereof, and mixtures thereof. In certain embodiments local anesthetics are commercially available from suppliers known to those skilled in the art.

In certain embodiments, local anesthetics include lidocaine (also known as lignocaine and the HCl salt form is known as zylocalne), bupivacaine, prilocalne, mepivacaine, etidocaine, ropivacaine, dibucaine, tetracaine, procaine, benzocaine, chloroprocaine, and pharmaceutically acceptable salts thereof, and mixtures thereof. In certain embodiments, the local anesthetic is lidocaine. A local anesthetic can be provided in its basic form. Lidocaine is 2-diethylamino-N-(2,6-dimethylphenyl)-acetamide and is available under the trademark Xylocaine®. Tetracaine is 2-dimethylaminoethyl-4-n-butylaminobenzoate and is available under the trademark Pontocaine®. Prilocalne is 2-propylamino-N-(2-tolyl)propionamide and is available under the trademark Citanest®. Procaine is 2-diethylaminoethyl p-aminobenzoate and is available under the trademark of Aminocaine®. Mepivacaine is 1-methyl-2-(2,6-xylylcarbomoyl)piperidine and is available under the trademark Carbocaine®. Benzocaine is 4-aminobenzoic acid ethyl ester and is available under the trademark Americaine®. Bupivacaine is 1-butyl-2-(2,6-cycylcarbomoyl)piperidine and is available under the trademark Marcaine®. Etidocaine is 2-ethylpropylamino-2,6-n-butyroxylidide and is available under the trademark Duranest®.

The amount of local anesthetic agent in topical formulations of the present disclosure can vary depending upon the particular formulation as well as the potency of the anesthetic agent employed. In certain embodiments, a topical composition can comprise from about 0.25 wt % to about 20 wt % of a local anesthetic agent. When the local anesthetic agent is lidocaine (free base or hydrochloride salt), a composition can comprise from about 1 wt % to about 10 wt % lidocaine. When local anesthetics such as procaine, chloroprocaine, tetracaine, mepivacaine, prilocalne, bupivacaine or etidocaine are used, a composition can comprise from about 0.5 wt % to about 10 wt % of the local anesthetic agent. In certain embodiments, compositions of the present disclosure can include more than one local anesthetic agent.

In certain embodiments, compositions of the present disclosure can comprise a vasoconstrictor to prolong the duration of local pain killing effect of a GABA analog prodrug and/or a local anesthetic agent. One suitable vasoconstrictor is epinephrine. In certain embodiments, compositions of the present disclosure can comprise an amount of vasoconstrictor ranging from about 1 part by weight per 10,000 to 200,000 parts by weight of a local anesthetic agent.

Topical delivery of a prodrug of gabapentin and/or a gabapentin analog can provide a high local drug concentration with minimal systemic exposure and reduced incidence of CNS-related side effects such as, for example, somnolence and dizziness. Pharmaceutical compounding techniques well known in the art can be employed for the formulation of local topical or transdermal compositions comprising gabapentin and/or pregabalin prodrugs. These include formulations such as lotions, creams, gels, microspheres, polymeric micelle formulations, liposomal formulations, transdermal patches, and the like.

Methods of the present disclosure comprise administering a prodrug of a GABA analog to a patient under conditions effective for treating or preventing local pain or discomfort. Thus, the present methods encompass reducing the number and/or frequency of experienced episodes of local pain or discomfort, reducing the severity of the experienced local pain or discomfort, or both. While the methods and compositions have utility in treating animals, the compositions can be useful in treating humans. More particularly, the methods and compositions disclosed herein are useful in treating local pain in humans that is caused by neuropathology or inflammation. As used herein the term “neuropathic pain” refers to pain syndromes known to be neuropathic (i.e., due to lesions or dysfunction in the nervous system) including certain relatively generalized syndromes, such as peripheral neuropathy, phantom pain, reflex sympathetic dystrophy, causalgia, central pain, syringomyelia, painful scar, and the like. Certain relatively localized syndromes are also considered to be neuropathic. Among these are specific neuralgias at any location of the body, head or face; diabetic, alcoholic, metabolic or inflammatory neuropathies; post herpetic neuralgias; post traumatic and post endodontic odontalgia; thoracic outlet syndrome; cervical, thoracic, or lumbar radiculopathies with nerve compression; cancer with nerve invasion; post traumatic avulsion injuries; post mastectomy pain, post thoracotomy pain; post spinal cord injury pain; post stroke pain; abdominal cutaneous nerve entrapments; primary tumors of neural tissues; arachnoiditis, and the like.

Other pain syndromes believed to have a neuropathic component include stump pain, fibromyalgia, regional sprains or strains (crushing injury), myofascial pain, psoriatic arthropathy, polyarteritis nodosa, osteomyelitis, burns involving nerve damage, AIDS related pain syndromes, and connective tissue disorders, such as systemic lupus erythematosis, systemic sclerosis, polymyositis, dermatomyositis, and the like.

Compositions and methods of the present disclosure can also be useful for treating or preventing local pain and discomfort caused by inflammatory conditions. Inflammatory conditions that can be treated by the disclosed methods and compositions include conditions of acute inflammation (e.g. trauma, surgery and infection) and chronic inflammation (e.g., arthritis and gout).

The relative high concentrations of drugs attainable by local administration, coupled with a lesser incidence of the side effects characteristic of systemic absorption, can produce particular benefits in treatment of local pain or discomfort associated with neuropathic or inflammatory pain conditions using the compositions and methods disclosed herein.

Prodrugs of GABA analogs and compositions of the present disclosure can be incorporated into compositions, formulations and dosage forms appropriate for topical application. Suitable compositions, formulations and dosage forms include ointments, creams, gels, lotions, pastes, sprays liposomes, micelles, microspheres, plasters, transdermal systems, and the like.

Ointments, as is well known in the art of pharmaceutical formulation, are semi-solid preparations that are typically based on petrolatum or other petroleum derivatives. The specific ointment base to be used, as will be appreciated by those skilled in the art, is one that will provide for optimum drug delivery, and, in certain embodiments, will provide for other desired characteristics as well, e.g., emolliency and the like. As with other carriers or vehicles, an ointment base should be inert, stable, nonirritating and nonsensitizing. As explained in Remington, “The Science and Practice of Pharmacy,” 19th Ed. (Easton, Pa., Mack Publishing Co., 1995), at pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Water-soluble ointment bases can also be prepared from polyethylene glycols of varying molecular weight; again, see Remington: The Science and Practice of Pharmacy for further information.

Creams are viscous liquids or semisolid emulsions, either oil-in-water or water-in-oil. Cream bases are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

Gels are semisolid, suspension-type systems. Single-phase gels contain organic macromolecules distributed substantially uniformly throughout the carrier liquid, which is typically aqueous, but also, can contain an alcohol such as ethanol or isopropanol and, optionally, an oil. Examples of organic macromolecules include crosslinked acrylic acid polymers such as the “carbomer” family of polymers, e.g., carboxypolyalkylenes that may be obtained commercially under the trademark Carbopol®. Other examples of useful organic macromolecules include hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing, stirring, or combinations thereof.

Lotions are preparations to be applied to the skin surface without friction, and are typically liquid or semi-liquid preparations in which solid particles, including the active agent, are present in a water or alcohol base. Lotions are usually suspensions of solids, and preferably, for the present purpose, comprise a liquid oily emulsion of the oil-in-water type. Lotions can be particularly appropriate for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

Pastes are semisolid dosage forms in which the active agent is suspended in a suitable base. Depending on the nature of the base, pastes are divided between fatty pastes or those made from single-phase aqueous gels. The base in a fatty paste is generally petrolatum, hydrophilic petrolatum or the like. Pastes made from single-phase aqueous gels generally incorporate carboxymethylcellulose or the like as a base.

Formulations may also include liposomes, micelles, and microspheres.

Liposomes are microscopic vesicles having a lipid wall comprising a lipid bilayer and can be used as drug delivery systems herein as well. Generally, liposome formulations are preferred for poorly soluble or insoluble pharmaceutical agents. Liposomal preparations for use in the compositions and methods of the present disclosure include cationic (positively charged), anionic (negatively charged) and neutral preparations. Cationic liposomes are readily available. For example, N-[1-2,3-dioleyloxy)propyl]-N,N,N-triethylammonium (DOTMA) liposomes are available under the trademark LIPOFECTIN (GIBCO BRL, Grand Island, N.Y.). Similarly, anionic and neutral liposomes are readily available, for example, from Avanti Polar Lipids (Birmingham, Ala.) or can be easily prepared using readily available materials. Such materials useful for preparing liposomes include phosphatidyl choline, cholesterol, phosphatidyl ethanolamine, dioleoylphosphatidyl choline (DOPC), dioleoylphosphatidyl glycerol (DOPG), dioleoylphosphatidyl ethanolamine (DOPE), and the like. These materials can also be mixed with DOTMA in appropriate ratios. Methods for making liposomes using these and other materials are well known in the art.

Micelles are known in the art as comprising surfactant molecules arranged so that the polar head groups form an outer spherical shell, while the hydrophobic, hydrocarbon chains are oriented towards the center of the sphere, forming a core. Micelles can form in an aqueous solution containing a sufficiently high surfactant concentration. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethylammonium chloride, dodecylammonium chloride, polyoxyl 8 dodecyl ether, polyoxyl 12 dodecyl ether, nonoxynol 10 and nonoxynol 30. Micelle formulations can be used in compositions of the present disclosure either by incorporation into the reservoir of a topical or transdermal delivery system, or into a formulation to be applied to the body surface.

Microspheres, similarly, may be incorporated into the disclosed formulations and transdermal systems. Like liposomes and micelles, microspheres essentially encapsulate a drug or drug-containing formulation. Microspheres are generally although not necessarily formed from lipids, preferably charged lipids such as phospholipids. Preparation of lipidic microspheres is well known in the art and described in the pertinent texts and literature.

For those GABA analog prodrugs and/or local anesthetic agents requiring a higher rate of skin or mucosal tissue penetration, a permeation enhancer may optionally be included in a topical composition of the present disclosure. It is desirable that a permeation enhancer should minimize the possibility of skin damage, irritation, and systemic toxicity. Examples of suitable permeation enhancers include, but are not limited to, ethers such as diethylene glycol monoethyl ether (available commercially as Transcutol®) and diethylene glycol monomethyl ether; surfactants such as sodium laurate, sodium lauryl sulfate, cetyltrimethylammonium bromide, benzalkonium chloride, Poloxamer (231, 182, 184) Tween (20, 40, 60, 80) and lecithin (U.S. Pat. No. 4,783,450); alcohols such as ethanol, propanol, octanol, benzyl alcohol, and the like; fatty acids such as lauric acid, oleic acid and valeric acid; fatty acid esters such as isopropyl myristate, isopropyl palmitate, methylpropionate, and ethyl oleate; polyols and esters thereof such as polyethylene glycol, and polyethylene glycol monolaurate (PEGML; see for example, U.S. Pat. No. 4,568,343); amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine; terpenes; alkanones; organic acids such as citric acid and succinic acid; AZONE and sulfoxides such as DMSO and C₁₀MSO.

A composition of the present disclosure can contain irritation-mitigating additives to minimize or eliminate the possibility of skin irritation or skin damage resulting from the prodrug, local anesthetic agent, a vasoconstrictor and/or a permeation enhancer included in the topical composition. Suitable irritation-mitigating additives include, for example, α-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine. An irritant-mitigating additive can be incorporated into a composition at a concentration effective to mitigate irritation or skin damage, in certain embodiments, less than about 20 wt %, and in certain embodiments less than about 5 wt %, of the total weight of the composition.

Various additives, known to those skilled in the art, may be included in topical compositions of the present disclosure. For example, solvents, including relatively small amounts of alcohol, may be used to solubilize a prodrug of a GABA analog and/or a local anesthetic agent. Other optional additives include opacifiers, antioxidants, fragrances, colorant, gelling agents, emulsifiers, thickening agents, stabilizers, surfactants, buffers, cooling agents (e.g., menthol) and the like. Other agents may also be added, such as antimicrobial agents, to prevent spoilage upon storage, i.e., to inhibit growth of microbes such as yeasts and molds. Examples of suitable antimicrobial agents include methyl and propyl esters of p-hydroxybenzoic acid (i.e., methyl and propyl paraben), sodium benzoate, sorbic acid, imidurea, and the like.

When applied to skin, a topical composition of the present disclosure can be covered with an occlusive or non-occlusive dressing, which may be porous or non-porous, so as to protect the composition from mechanical removal during the period of treatment, e.g. a plastic film food wrap or other non-absorbent film. Various inert coverings may be employed. Non-woven or woven coverings may be employed, particularly elastomeric coverings, which allow for heat and vapor transport. These coverings can allow for cooling of the pain site, which can provide for greater comfort, while protecting the composition from mechanical removal.

Compositions of the present disclosure can be included in a skin-contacting plaster or patch, i.e., a transdermal system, wherein the composition is contained within a material, e.g., a drug reservoir layer, that can be affixed to the skin. In certain embodiments, the active agent or agents can be contained in a drug reservoir layer underlying an upper backing layer. The system may contain a single reservoir, or it may contain multiple reservoirs. In these systems the active agent(s) may be formulated with the adhesive used to adhere the system to the skin. The system can include a backing layer which functions as the primary structural element of the transdermal system and can provide the system with flexibility and, preferably, occlusivity. The material used for the backing layer can be inert and incapable of absorbing the components of the composition contained within the system. The backing can comprise a flexible elastomeric material that can serve as a protective covering to prevent loss of components of the composition via transmission through the upper surface of the patch, and in certain embodiments can impart a degree of occlusivity to the system, such that the area of the body surface covered by the patch becomes hydrated during use. The material used for the backing layer can permit the system to follow the contours of the skin and be worn comfortably on areas of skin such as at joints or other points of flexure, that are normally subjected to mechanical strain with little or no likelihood of the device disengaging from the skin due to differences in the flexibility or resiliency of the skin and the system. The materials used as the backing layer can be either occlusive or permeable, as noted herein, although occlusive backings are preferred, and are generally derived from synthetic polymers (e.g., polyester, polyethylene, polypropylene, polyurethane, polyvinylidine chloride, and polyether amide), natural polymers (e.g., cellulosic materials), or macroporous woven and nonwoven materials. In some systems, the upper backing layer can be an adhesive overlay that secures the system to the skin. The adhesive overlay can be sized such that it extends beyond the drug reservoir so that adhesive on the overlay contacts the skin surrounding the drug reservoir. The skin-contacting side of the overlay can be coated with a skin-compatible adhesive. The prodrug and anesthetic agent can be contained in a separate drug reservoir layer, or within the coated adhesive, e.g., with the aid of a cosolvent, or a combination of cosolvents, such as propylene glycol, glycerin, methyl salicylate, glycol salicylate, and/or the like. The particular choice of adhesive is not critical, there being a wide variety of physiologically acceptable adhesives which can maintain the system in contact with the skin for the necessary period of treatment.

In certain embodiments, the reservoir of a transdermal delivery system can comprise a polymeric matrix of a pharmaceutically acceptable adhesive material that serves to affix the system to the skin during prodrug delivery; typically, the adhesive material is a pressure-sensitive adhesive that is suitable for long-term skin contact, and which is physically and chemically compatible with the components of the composition, e.g., the GABA analog prodrug, the local anesthetic agent, and any carriers, vehicles or other additives that are present. Examples of suitable adhesive materials include, but are not limited to, polyethylenes; polysiloxanes; polyisobutylenes; polyacrylates; polyacrylamides; polyurethanes; plasticized ethylene-vinyl acetate copolymers; and tacky rubbers such as polyisobutene, polybutadiene, polystyrene-isoprene copolymers, polystyrene-butadiene copolymers, and neoprene (polychloroprene). In certain embodiments, the adhesive is chosen from a polyisobutylene.

During storage and prior to use, the laminated structure of a transdermal delivery system can include a release liner. Immediately prior to use, the release layer can be removed from the system so that the system may be affixed to the skin. The release liner can be made from a material that is impermeable to the components of the composition, and can be a disposable element which serves only to protect the device prior to application. In certain embodiments, a release liner can be formed from a material impermeable to the components of the composition and which can be easily stripped from the transdermal delivery system prior to use.

In certain embodiments, the composition-containing reservoir and skin contact adhesive can be present as separate and distinct layers, with the adhesive underlying the reservoir. In such a case, the reservoir may be a polymeric matrix as described herein. Alternatively, the reservoir may comprise a liquid or semisolid composition contained within a closed compartment or “pouch,” or it may be a hydrogel reservoir, or may take some other form. In certain embodiments, the reservoir can comprise a hydrogel. As will be appreciated by those skilled in the art, hydrogels are macromolecular networks that absorb water and thus swell but do not dissolve in water. Hydrogels can comprise hydrophilic functional groups that provide for water absorption and crosslinked polymers that provide aqueous insolubility. Hydrogels can comprise crosslinked hydrophilic polymers such as polyurethane, polyvinyl alcohol, polyacrylic acid, polyoxyethylene, polyvinylpyrrolidone, poly(hydroxyethyl methacrylate) (poly(HEMA)), or a copolymer or mixture thereof. In certain embodiments, hydrophilic polymers forming a hydrogel are copolymers of HEMA and polyvinylpyrrolidone.

Additional layers, e.g., intermediate fabric layers and/or rate-controlling membranes, can also be present in any of the drug delivery systems disclosed herein. Fabric layers may be used to facilitate fabrication of the system, and/or to control the rate at which a component of a formulation permeates out of the system. A rate-controlling membrane can be included in the system on the skin side of one or more of the drug reservoirs. The materials used to form a rate-controlling membrane can be selected to limit the flux of one or more components of a composition. Representative materials useful for forming rate-controlling membranes include polyolefins such as polyethylene and polypropylene, polyamides, polyesters, ethylene-ethacrylate copolymer, ethylene-vinyl acetate copolymer, ethylene-vinyl methylacetate copolymer, ethylene-vinyl ethylacetate copolymer, ethylene-vinyl propylacetate copolymer, polyisoprene, polyacrylonitrile, ethylene-propylene copolymer, and the like.

A transdermal delivery system can be applied to the site of pain or discomfort and may be of any convenient and/or appropriate size to cover or partially cover the affected area. A transdermal delivery system can be provided in large sheets, e.g. 30×50 cm sheets, which may be cut to an appropriate size or in a variety of sizes. In the case of pre-manufactured transdermal delivery systems, the system can have a preset skin contact area ranging, for example, from about 1 cm² to about 200 cm², in certain embodiments, from about 1 cm² to about 100 cm², and in certain embodiments from about 1 cm² to about 50 cm². Larger patches can be used for treating larger areas of local pain or discomfort, while smaller patches can be used for treating smaller areas of pain or discomfort. Once a system is applied, it can be left in place for up to about 7 days, during which time significant relief of local pain and discomfort can be achieved. In some patients, relief can be maintained after removal of the system. When pain returns after removal of a system, another system may be applied to the same site.

Transdermal delivery systems can be fabricated using conventional coating and laminating techniques known in the art. For example, adhesive matrix systems can be prepared by casting a fluid admixture of adhesive, drug and vehicle onto the backing layer, followed by lamination of the release liner. Similarly, the adhesive mixture can be cast onto the release liner, followed by lamination of the backing layer. Alternatively, the drug reservoir can be prepared in the absence of a composition, and then loaded by “soaking” in the composition. Transdermal delivery systems can be fabricated by solvent evaporation, film casting, melt extrusion, thin film lamination, die cutting, or the like. In certain embodiments, compositions comprising a GABA analog prodrug and anesthetic agent can be incorporated into a system during system manufacture rather than after system manufacture.

The concentration of a GABA analog prodrug in the composition, or in the case of a transdermal delivery system, the concentration of the GABA analog prodrug in the reservoir of the system, can vary a great deal, and will depend on a variety of factors, including the type and severity of pain being treated, the desired duration of pain relief, possible adverse reactions, the effectiveness of the GABA analog prodrug, and other factors within the particular knowledge of the patient and physician. In certain embodiments, compositions of the present disclosure can comprise an amount of GABA analog prodrug ranging from about 0.5 wt % to about 50 wt %, in certain embodiments from about 0.5 wt % to about 5 wt %, and in certain embodiments from about 5 wt % to about 20 wt %.

Methods of treating or preventing local pain or discomfort of the present disclosure can comprise topically administering to the site of local pain or discomfort a therapeutically effective amount of a GABA analog prodrug, optionally with a local anesthetic agent, to a patient in need of such treatment. A GABA analog prodrug and optionally a local anesthetic agent, or a pharmaceutical composition containing same, can be administered topically to the skin or mucosa, for example, oral mucosa, rectal mucosa, nasal mucosa, and the like. Topical administration of a GABA analog prodrug to a site of local pain or discomfort includes administering a topical composition of the present disclosure.

The amount of GABA analog prodrug that will be effective in the treatment of local pain or discomfort in a patient can depend on, among other factors, the specific cause of the pain (e.g., neuropathic or pain caused by inflammation), the subject being treated, the weight of the subject, the severity of the pain or underlying (e.g., neuropathic) condition which is causing the pain, the manner of administration, the formulation and the judgment of the prescribing physician. The amount of GABA analog prodrug that will be effective in the treatment of local pain or discomfort in a patient can be determined by standard clinical techniques known in the art. In addition, in vitro or in vivo assays may be employed to identify optimal dosage ranges. Topical compositions of the present disclosure can be adapted to be administered to a patient no more than twice per day, and in certain embodiments, only once per day. When a composition of the present disclosure is administered using a transdermal delivery system, the dosing can be no more than once per day, and in certain embodiments, less than 3 times per week. Dosing may be provided alone or in combination with other drugs and may continue as long as required for effective treatment of the pain.

Suitable dosage ranges for topical administration can depend on the potency of the particular GABA analog drug (once cleaved from the promoiety) and the area of skin or mucosa in which the local pain or discomfort is experienced. In certain embodiments, a therapeutically effective dose for treating local pain or discomfort can range from about 0.005 mg to about 100 mg of GABA analog prodrug per cm² of skin or mucosa surface per day, and in certain embodiments from about 0.05 mg to about 20 mg of prodrug per cm² of skin or mucosa surface per day. In certain embodiments, the GABA analog prodrug is a prodrug of gabapentin or pregabalin. Dosage ranges may be readily determined by methods known to the skilled artisan.

Suitable dosage ranges for administration of the optional local anesthetic agent can depend upon the potency of the particular local anesthetic agent, and in certain embodiments can range from about 1 mg to about 100 mg of local anesthetic agent per cm of skin or mucosa surface per day, and in certain embodiments, from about 10 mg to about 80 mg of local anesthetic agent per cm² of skin or mucosa surface per day. In certain embodiments, the local anesthetic agent is lidocaine, and in certain embodiments, lidocaine in free base form. The amount of lidocaine in a typical composition of the present disclosure can range from about 1 wt % to about 25 wt %.

All publications and patents cited herein are incorporated herein by reference in their entirety.

### EXAMPLES

The following examples describe in detail preparation of compounds and compositions disclosed herein and assays for using compounds and compositions disclosed herein. It will be apparent to those of ordinary skill in the art that many modifications, both to materials and methods, may be practiced.

### Determination of Permeability of Gabapentin and Gabapentin Prodrugs in a Cultured Human Epithelial Cell Monolayer Assay

The following comparative test was run to determine the permeability of gabapentin and a prodrug of gabapentin across a cultured monolayer of human mucosal tissue cells (intestinal epithelial cells). Intestinal epithelial cells have an apical side (i.e., the side normally facing the gut lumen) and a basolateral side (i.e., the side normally facing the patient's internal blood carrying tissues). Both the permeability of the compounds through the monolayer of cells from apical to basolateral (A to B) and basolateral to apical (B to A) were measured. This test procedure has been demonstrated to be useful in determining the ability of a compound to permeate through human skin (see Gyurosiova et al., Pharm. Res., 2002 February, 19(2), pp. 162-168.

Caco-2 cells were obtained from the ATCC (Manassas, Va.) and cultured as indicated by the supplier. Caco-2 cells were seeded into 24-well transwell plates with 3 μm filters (Corning/Costar, Acton, Mass.) at a density of 500,000 cells/well and allowed to differentiate in the transwell plates for 21 days. The test compounds were dissolved into either pH 6.5 (apical MES (4-morpholineethane sulfonic acid) buffer) or pH 7.4 (basolateral HBSS (Hanks balanced salt solution) buffer) at concentrations of 100 to 200 μM and added to the appropriate chambers. Samples were removed from the receiving chambers at various times and the permeability was measured by determining the concentration of prodrug and gabapentin (produced by esterase cleavage within the epithelial cells) by LC/MS/MS (liquid chromatography/mass spectroscopy/mass spectroscopy). Apparent permeability coefficients (“Pₐₚₚ”) were calculated by standard methods (B. H. Stewart et al., Pharm. Res., 1995, 12, pp. 693-699). Integrity of the monolayer was confirmed by determining the permeability of 3H-inulin. If greater than 0.5% of the inulin was detected in the receiving chamber, the transwells were discarded. The apparent permeability coefficient, Pₐₚₚ, of 1-{[(α-isobutanoyloxyethoxy)carbonyl]-aminomethyl}-1-cyclohexane acetic acid from the basolateral to the apical side of Caco-2 cell monolayers was significantly higher than the Pₐₚₚ for gabapentin as shown in the data presented in Table 1.

TABLE 1
Permeability Coefficients (Pₐₚₚ) for 1-{[(α-
isobutanoyloxyethoxy)carbonyl]-aminomethyl}-1-
cyclohexane acetic acid or Gabapentin Across
Caco-2 Monolayers*

Pₐₚₚ
(cm/sec) × 10⁶

| Test Compound | A to B^{a} | B to A |
|---|---|---|
| 1-{[(α-isobutanoyloxyethoxy) | 31 | 5.7 |
| carbonyl]-aminomethyl}-1- | | |
| cyclohexane acetic acid | | |
| Gabapentin | 3.2 | 3.4 |

*Compounds were applied to the donor compartment at 100-200 μM and incubated for 1 hr at 37° C.
^{a}A—apical; B—basolateral.

### Determination of Passive Permeability of Gabapentin and Gabapentin Prodrugs in a Parallel Artificial Membrane Assay

Artificial membranes were prepared by adding 4 μL of 2% (w/v) dioleoylphosphatidyl-choline in dodecane onto the hydrophobic filters (0.45 μM polyvinylidene fluoride) on the base of the wells of a 96-well donor plate (Millipore, Bedford, Mass.). Gabapentin or a gabapentin prodrug (150 μL of 50 μM solution in 0.1 M Tris buffer, pH 6.5 or pH 7.4) was added to the donor wells in triplicate. The gabapentin prodrug used was 1-{[(α-isobutanoyloxyethoxy)carbonyl]-aminomethyl}-1-cyclohexane acetic acid. The plate was placed onto a 96-well acceptor plate (Agilent, Wilmington, Del.), in which each well contained 400 μL 0.1 M Tris, pH 7.4. Following incubation for two hours at room temperature, samples of the donor and receiver chambers were removed for analysis by LC/MS/MS. The permeability coefficient through the artificial membrane (“Pₐₘ”) was calculated using standard methods (Sugano et al., Int. J. Pharm., 2001, 228, pp. 181-188). The artificial membrane permeability coefficient, Pₐₘ, of 1-{[(α-isobutanoyloxyethoxy)carbonyl]-aminomethyl}-1-cyclohexane acetic acid was about 5 to 30 times higher than the Pam for gabapentin at the two test pH values as shown in Table 2.

TABLE 2
Effect of Donor Compartment pH on Permeability Coefficients
(Pₐₘ) of Various Compounds in the Parallel Artificial
Membrane Permeability Assay

| | |
|---|---|
| | Pₐₘ |
| Test | (cm/sec) × 10⁶ |

| Compound | pH 6.5^{a} | pH 7.4^{a} |
|---|---|---|
| 1-{[α- | 0.65 | 0.095 |
| isobutanoyloxyethoxy)carbonyl]- | | |
| aminomethyl}-1-cyclohexane | | |
| acetic acid | | |
| Gabapentin | 0.02 | 0.026 |

^{a}Indicates pH of the donor chamber.

### Administration of 1-{[(α-Isobutanoyloxyethoxy)carbonyl]-aminomethyl}-1-Cyclohexane Acetic Acid for the Treatment of Neuropathic Pain

A placebo-controlled clinical trial is conducted to assess the effects of the prodrug 1-{[(α-isobutanoyloxyethoxy)carbonyl]-aminomethyl}-1-cyclohexane acetic acid alone and in combination with the topical anesthetic lidocaine on sensory symptoms in patients with neuropathic pain, according to the general method of M. C. Rowbotham and H. L. Fields (Pain, 1989, 38:297-301). Briefly, sixty patients with post-herpatic neuralgia, a condition that typifies neuropathic pain, are randomized and treated with ointments containing the prodrug alone, the prodrug in combination with lidocaine, or placebo.

Prior to topical application, the painful area to be treated is marked and photographed based on the subject's report of (1) the borders of the area of sensory abnormality, and (2) the area of greatest pain. Ointment comprising the prodrug and local anesthetic is then applied in the amount of 1-2 g of ointment per 10 cm² of skin. Subjects are observed for the first 6 hours following application. The subjects make ratings of pain, pain relief, and side effects at 6 hours, 9 hours and 12 hours after initial application of the ointment.

Pain intensity is assessed using a horizontal 100 mm visual analog scale (“VAS”). The subject indicates the severity of his or her pain with a mark along the line between “no pain” (0 mm) and “worst pain imaginable” (100 mm). Prior to application, VAS scores are obtained 3 times over a 45-minute period; once before quantitative sensory testing (“QST”) and two times following QST. After application, VAS scores are obtained at 30 minutes, 1 hour, 2 hours, 4 hours, 6 hours, 9 hours, and 12 hours.

Pain relief is assessed using a category scale consisting of 6 sentences indicating that: the pain is increasing (score 0), “no” pain relief (1), “slight” pain relief (2), “moderate” pain relief (3), “a lot” of pain relief (4), and “complete” relief of pain (5). As the scale is designed to assess changes only, there is no baseline pre-application rating. After topical application, category relief scores are obtained at 30 minutes, 1 hour, 2 hours, 4 hours, 6 hours, 9 hours, and 12 hours.

A statistical analysis of the data obtained is conducted using the method of analysis of variance whenever possible. This is accomplished using the Statistical Analysis System (SAS) v. 6.04, under the procedure General Linear Models. An overall F-test is conducted to determine if there are differences among the three treatments. Additionally, pairwise contrast tests between treatments are performed to evaluate the statistical significance between pairs of treatments. The difference between two treatments (F-test) is considered statistically significant if both the overall and pairwise p-values are less than or equal to 0.05. For pain intensity VAS scores and QST data, the pairwise comparisons are made at individual time points in addition to the overall F-test. A positive result for the prodrug or the prodrug combined with lidocaine is associated with reduced symptoms on all rating scales when compared with the placebo.

While certain embodiments have been shown and described, various modifications and substitutions may be made thereto without departing from the spirit and scope of the invention. Accordingly, it is to be understood that the present invention has been described by way of illustration and not limitation.

1. A topical composition for treating or preventing pain or discomfort comprising:
a compound chosen from Formula (1) and Formula (2): 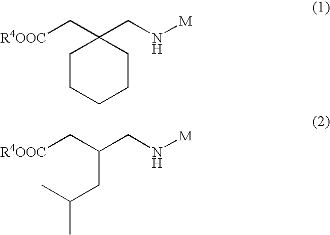
wherein:
R⁴ is chosen from hydrogen and a labile ester-forming group chosen from C₁₋₆ alkyl, benzyl, and phenyl groups that become removed in the body of a subject;
M is a moiety that becomes removed in the body of a subject and which increases skin permeability of the compound to a level greater than the skin permeability of a modified compound formed by replacing either M or both M and R⁴ with hydrogen;
or a pharmaceutically acceptable salt, hydrate or solvate thereof; and
a pharmaceutically acceptable vehicle.

2. The composition of claim 1, wherein M is a moiety of Formula (3): 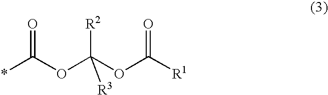
wherein:
R¹ is chosen from C₁₋₆ alkyl, and 1,1-diethoxyethyl; and
R² and R³ are independently chosen from hydrogen, and C₁₋₆ alkyl.

3. The composition of claim 1, wherein M is a moiety of Formula (3): 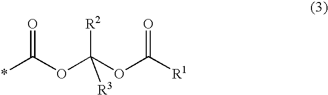
wherein:
R¹ is chosen from methyl, ethyl, propyl, isopropyl, butyl, isobutyl, sec-butyl, tert-butyl, pentyl, isopentyl, sec-pentyl, neopentyl, and 1,1-diethoxyethyl; and
R² and R³ are independently chosen from hydrogen, methyl, ethyl, propyl, isopropyl, butyl, isobutyl, and sec-butyl.

4. The composition of claim 1, further comprising a local anesthetic agent.

5. The composition of claim 4, wherein the local anesthetic agent is chosen from lidocaine, procaine, chloroprocaine, tetracaine, mepivacaine, prilocaine, bupivacaine, etidocaine, ropivacaine, dibucaine, benzocaine, and pharmaceutically acceptable salts thereof.

6. The composition of claim 4, wherein the local anesthetic agent is lidocaine.

7. The composition of claim 1, wherein the compound is 1-{[α-isobutanoyloxyethoxy)carbonyl]-aminomethyl}-1-cyclohexane acetic acid.

8. The composition of claim 4, wherein the compound is 1-{[α-isobutanoyloxyethoxy)carbonyl]-aminomethyl}-1-cyclohexane acetic acid and the local anesthetic agent is lidocaine.

9. The composition of claim 1, wherein the composition is in the form of a topical gel, ointment or cream.

10. The composition of claim 1, wherein the composition is included in a transdermal delivery system.

11. The composition of claim 1, wherein the pain is neuropathic pain.

12. The composition of claim 1, wherein the compound is a substantially pure optical isomer of Formula (4):

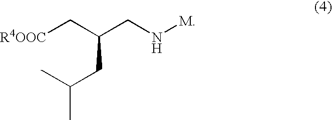

13. The composition of claim 1, wherein M is a moiety which increases the artificial membrane permeability coefficient of the compound to a level that is at least 5 times greater than the artificial membrane permeability coefficient of the modified compound.

14. The composition of claim 1, wherein M is a moiety which increases the apparent permeability coefficient of the compound to a level that is at least 50% greater than the apparent permeability coefficient of the modified compound.

15. A method of treating or preventing pain or discomfort in a subject having a site of local pain or discomfort, comprising locally administering to the site a therapeutically effective amount of a compound chosen from Formula (1) and Formula (2): 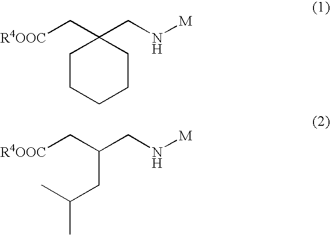
wherein:
R⁴ is chosen from hydrogen and a labile ester-forming group chosen from C₁₋₆ alkyl, benzyl, and phenyl groups that become removed in the body of a subject;
M is a moiety that becomes removed in the body of the subject and which increases skin permeability of the compound to a level greater than the skin permeability of a modified compound formed by replacing either M or both M and R⁴ with hydrogen;
or a pharmaceutically acceptable salt, hydrate or solvate thereof.

16. The method of claim 15, wherein M is a moiety of Formula (3): 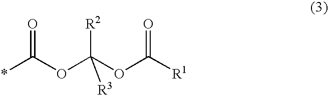
wherein:
R¹ is chosen from C₁₋₆ alkyl, and 1,1-diethoxyethyl; and
R² and R³ are independently chosen from hydrogen, and C₁₋₆ alkyl.

17. The method of claim 15, wherein M is a moiety of Formula (3): 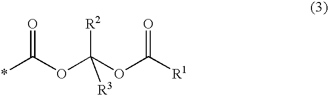
wherein:
R¹ is chosen from methyl, ethyl, propyl, isopropyl, butyl, isobutyl, sec-butyl, tert-butyl, pentyl, isopentyl, sec-pentyl, neopentyl, and 1,1-diethoxyethyl; and
R² and R³ are independently chosen from hydrogen, methyl, ethyl, propyl, isopropyl, butyl, isobutyl, and sec-butyl.

18. The method of claim 15, comprising co-administering the compound with a local anesthetic agent.

19. The method of claim 18, wherein the local anesthetic agent is chosen from lidocaine, procaine, chloroprocaine, tetracaine, cocaine, mepivacaine, prilocalne, bupivacaine, and etidocaine, and pharmaceutically acceptable salts thereof.

20. The method of claim 18, wherein the local anesthetic agent is lidocaine.

21. The method of claim 15, wherein the compound is 1-{[α-isobutanoyloxyethoxy)carbonyl]-aminomethyl}-1-cyclohexane acetic acid.

22. The method of claim 18, wherein the compound is 1-{[α-isobutanoyloxyethoxy)carbonyl]-aminomethyl}-1-cyclohexane acetic acid and the local anesthetic agent is lidocaine.

23. The method of claim 15, wherein the compound is in the form of a topical gel, ointment or cream.

24. The method of claim 15, wherein the compound is included in a transdermal delivery system.

25. The method of claim 15, wherein the pain is neuropathic pain.

26. The method of claim 15, wherein the compound is a substantially pure optical isomer of Formula (4):

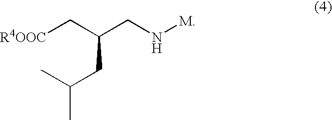

27. The method of claim 15, wherein M is a moiety which increases the artificial membrane permeability coefficient of the compound to a level that is at least 5 times greater than the artificial membrane permeability coefficient of the modified compound.

28. The method of claim 15, wherein M is a moiety which increases the apparent permeability coefficient of the compound to a level that is at least 50% greater than the apparent permeability coefficient of the modified compound.

29. The method of claim 15, wherein the method produces a local analgesic effect.
